(19) United States
(12) Patent Application Publication
(10) Pub. No.: US 2008/0103201 A1
(43) Pub. Date: May 1, 2008

(54) Novel alpha-Glucosidase inhibitor from Tabernaemontana dichotoma

(76) Inventors: Mirihanage Don Jayantha Wijayabandara, (US); Shamsun Nahar Khan, (US); Muhammad Iqbal Choudhary, (US)

Correspondence Address: SARFARAZ K. NIAZI, 20 RIVERSIDE DRIVE, DEERFIELD, IL, 60015, US

(21) Appl. No.: 11/553,465

(22) Filed: Oct. 26, 2006

(51) Int. Cl. A61K 31/22; C07C 69/74

(52) U.S. Cl. 514/548; 560/6

(57) ABSTRACT

This invention is directed to a novel pharmacological property of β-Amyrin acetate derived from Tabernaemontana dichotoma as a potent α-glycosidase inhibitor, which has not been reported before. The present discovery also provides for the use of this compound as a new oral hypoglycemic drug in the treatment of type II diabetes being approximately 35 times potent compared with Acarbose, a standard drug widely prescribed to type II diabetes patients.

### BACKGROUND

Glucosidase enzymes are involved in several biological processes such as the intestinal digestion, the biosynthesis of glycoproteins and the lysosomal catabolism of the glycoconjugates (Homonojirimycin isomers and N-alkylated homonojirimycins: structural and conformational basis of inhibition of glycosidases. Asano N, Nishida M, Kato A, Kizu H, Matsui K, Shimada Y, Itoh T, Baba M, Watson A A, Nash R J, Lilley P M, Watkin D J, Fleet G W., J Med Chem. 1998 Jul. 2; 41(14):2565-71). Intestinal α-glucosidases are involved in the final step of the carbohydrate digestion to convert these into monosaccharide, which are absorbed from the intestine.

Non insulin-dependant diabetes (NIDDM) or type II diabetes is expanding at an alarming rate around the world for a multitude of reasons including the sedentary lifestyle and obesity. The number of people with diabetes is expected to rise worldwide from the current estimated at 190 million to over 220 million by 2010 and 300 million by 2025. In Sri Lanka there are over 1.5 million diabetes patients. The complications associated with diabetes include retinopathy, neuropathy and nephropathy whose treatment and management places a large financial burden, specially on populations which do not have a well-developed healthcare support system.

D-Glucose and insulin levels of plasma are usually high in diabetics especially after food ingestion, and reducing intestinal carbohydrate absorption, such as monosaccharides, which are hydrolyzed by β-amylase and α-glycosidase, is one way to control disorders of carbohydrate metabolism. Therefore, α-glucosidase inhibitors are suggested to be valuable aids in the treatment of diabetes. They act by delaying the digestion and absorption of carbohydrates, thereby inhibiting postprandial hyperglycemia and hypernulinemia.

Glucosidase inhibitors are of particular interest in the development of potential pharmaceuticals such as antidiabetics, antitumour, antivirals, antibacterials.

As a result of the catalysis produced by α-glucosidase enzyme in the final step in the digestive process of carbohydrates, its inhibitors can retard the uptake of dietary carbohydrates and suppress postprandial hyperglycemia, and could be useful to treat diabetic and/or obese patients [Novel α-glucosidase Inhibitors with a tetrachlorophthalimide Skeleton., S. Sou, S. Mayumi, H. Takahashi, R. Yamasak, S Kadoya, M. Sodeoka, and Y. Hashimoto, Bioorg. Med Chem. Lett., 2000, 10, 1081]

The α-glucosidase inhibitors are effective in lowering the insulin release, insulin requirement and some can lower plasma lipids. The acarbose is a very widely prescribed drug in the management of the type II diabetes and recently a U.S. Pat. No. 6,387,361 to Rosner describes the use of acarbose in the treatment of obesity. According to the criteria issued by WHO (World Health Organization) based on a glucose tolerance test, diabetes mellitus and impaired glucose tolerance (hereinafter sometimes referred to as IGT) are distinguished by the fasting blood glucose level and the blood glucose level 2 hours after glucose loading. Patients with IGT have high blood glucose levels compared to those of patients with diabetes mellitus, and are reported to be at increased risk of developing diabetes mellitus and complications of arteriosclerotic diseases. In particular, it is known that patients with IGT who have blood glucose levels of 170 mg/dl or above at 2 hours following glucose loading, i.e., patients with high-risk IGT, may develop diabetes mellitus at a high rate [Diabetes Frontier, p. 136, 1992]. With regard to voglibose which is an α-glucosidase inhibitor, there are reports of studies on effects of voglibose for insulin-resistant IGT and diabetes [Yakuri-to-Chiryo (Japanese Pharmacology & Therapeutics), 24 (5):213 (1996); Metabol. Exp. Clin., 45:731, 1996]. Voglibose (AO-128) is also known to have effects of lowering blood glucose level and improving glucose tolerance in rats [Yakuri-to-Chiryo (Japanese Pharmacology & Therapeutics), 19 (11):161 (1991); Journal of Nutrition Science and Vitaminology, 45 (1): 33 (1992)]. On the contrary, it has also been reported that the effect of voglibose in improving glucose tolerance could not be verified in human [Rinsho-Seijinbyo, 22 (4): 109 (1992)]. An antibiotic pradimicin Q as α-glucosidase inhibitor is described in the U.S. Pat. No. 5,091,418 to Swada.

In addition, they have also been used as antiobesity drugs, fungistatic compounds, insect antifeedents, antivirals and immune modulators [Glycosidase inhibitors and their chemotherapeutic value, Part 1. el Ashry E S, Rashed N, Shobier A H., Pharmazie. 2000 April; 55(4):251-620]. The antiviral activity due to inhibition of α-glucosidase results form abnormal functionality of glycoproteins because of incomplete modification of glycans. Suppression of this process is the basis of antiviral activity [A glucosidase-Inhibitors as potential broad based antiviral agents, Anand Mehta, Nicole Zitzmann, Pauline M. Rudd, Timothy M. Block, Raymond A Dwek, Febs Letters 430 (1998)17-22] and decrease in growth rate of tumors [inhibition of experimental metastasis by an alpha-glucosidase inhibitor, 1,6-epi-cyclophellitol. Atsumi S, Nosaka C, Ochi Y, Iinuma H, Umezawa K. Cancer Res. 1993 Oct. 15; 53(20):4896-9]. The α-glucosidase inhibitor N-(1,3-dihydroxy-2-propyl)valiolamine is described as a promoter of calcium absorption in the U.S. Pat. No. 5,036,081.

In the present invention is reported a surprising invention when it was discovered that the β-amyrin acetate derived from Tabernaemontana dichotoma has a very potent α-glycosidase inhibitory activity which has not been known. The present discovery also provides for the use of this compound as a new oral hypoglycemic drug in the treatment of type II diabetes. β-Amyrin acetate showed approximately 35 times potent compared with Acarbose, a clinically used standard drug widely prescribed to type II diabetes patients. The compound of this invention reduced post-prandial blood glucose concentration in rats.

### BRIEF DESCRIPTION OF THE SEVERAL VIEWS OF THE DRAWING

FIG. 1 Structure of β-amyrin acetate (12-Oleanen-3-yl acetate), Compound I; R=α-H, β-OCOCH₃.

### DETAILED DESCRIPTION

The latex of Tabernaemontana dichotorma was collected in June 2006 from plants in Hanwella in the western province of Sri Lanka. The latex was obtained from twigs and leaves by breaking repeatedly and collecting the white milky exudates (0.2 litersl) directly in to 50% aqueous ethanol solution. The latex solution was centrifuged and the resulting coagulum (residue) was refluxed with methanol for 2 hours. The methanol solution was filtered and the filtrate was concentrated under vacuum to yield a white residue (0.5 g) which was subjected to silica gel column chromatography. Elution of the column with hexane:ethyl acetate (95:5) afforded four fractions with following fraction weights after solvent removal: A (150 mg), B (100 mg), C (150 mg) D (100 mg)]. Purification of fraction A by TLC using hexane:Ethyl acetate (95:5) yielded β-amyrin acetate (140 mg) (Compound I).

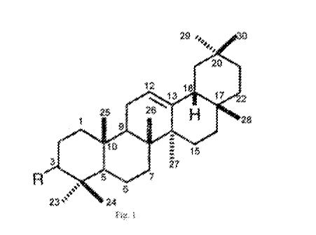

The structure of β-amyrin acetate (Compound I) was elucidated by comparison of its physical data (melting point) and spectroscopic data (¹H-, ¹³C-NMR, and Mass Spectra) with literature (Shunyo Matsunga, Reiko Tanaka and Masao Akagi (1988), Triterpenoids from Euphorbia Maculata), Phytochemistry, Vol 27, No. 2, pp. 535-537). FIG. 1 shows the structure of β-Amyrin acetate for the first time derived from Tabernaemontana dichotoma.

[FIG. 1]

General Analytical Instrumentation: TLC: Kieselgel F₂₅₄ (0.25 mm: Merck). Column chromatography (CC): silica gel (70-230 mesh; Merck), flash chromatography (FC): silica gel (230-400 mesh; Merck). Optical rotation: Jasco DIP-360 digital polarimeter. UV Spectra: Hitachi-UV-3200 spectrophotometer. IR spectra: Jasco-320-A spectrophotometer. ¹H-NMR, ¹³C-NMR, COSY, HMQC and HMBC Spectra: Bruker spectrometer, EI-MS and FAB-MS spectra: JMS-HX-110 spectrometer.

β-Amyrin acetate: Melting point. 243-245° C., MS; m/e (rel. %) 468 [M]⁺(70), 408[M-HOAc]⁺(10), 218(100), 203(59.7), 189(74).

β-Amyrin acetate: ¹HNMR(400 MHz,δ): 0.770 (s,H₃-23), 0.780 (s,H₃-24), 0.961 (s,H₃-25), 0.993 (s,H₃-26), 1.027 (s,H₃-27), 0.848 (s,H₃-28), 0.857 (s,H₃-29), 0.895 (s,H₃-30), 2.023 (s,H₃—OCOCH₃), 4.48 (H₁ d.d J=8.04, 5.9 Hz, H-3α) 5.11 (H1, t,J=3.5 Hz, H-12) β-Amyrin acetate: ¹³CNMR: 124.3 (CH), 140(q), 170.95 (q), 80 (q).

Activity Testing

α-Glucosidase (E.C.3.2.1.20) enzyme inhibition assay was performed according to the slightly modified method of Matsui et al. α-glucosidase (E.C.3.2.1.20) from Saccharomyces species, purchased from Wako Pure Chemical Industries Ltd. (Wako 076-02841). The enzyme inhibition was measured spectrophotometrically at pH 6.9 and at 37° C. using 0.7 mM p-nitrophenyl-α-D-glucopyranoside (PNP-G) as a substrate and 500 m units/mL enzyme, in 50 mM sodium phosphate buffer containing 100 mM NaCl. 1-Deoxynojirimycin (0.425 mM) and acarbose (0.78 mM) were used as positive control. The increment in absorption at 400 nm, due to the hydrolysis of PNP-G by α-glucosidase, was monitored continuously on microplate spectrophotometer (Spectra Max Molecular Devices, USA).) [T. Matsui, C. Yoshimoto, K. Osajima, T. Oki, and Y. Osajima. Biosci. Biotech. Biochem., 1996, 60, 2019].

TABLE 1
Result of In vitro quantitative studies on compounds 1 and
reference standard of known α-glucosidase inhibitors.

| Name of Substance | IC₅₀ ± SEM [μM] |
|---|---|
| β-Amyrin acetate | 22.27 ± 0.112 |
| Deoxynojirimycin (positive control for | 425 ± 8.14 |
| α-glucosidase) | |
| Acarbose (positive control for α- | 780 ± 0.028 |
| glucosidase) | |

1-3. (canceled)

4 . A method for the treatment of diabetes mellitus comprising of administration of a therapeutically effective amount of β-amyrin acetate to humans and animals.

5. As claimed in claim 4, where the said β-amyrin acetate is administered in a pharmaceutically elegant dosage form.

6. A pharmaceutical composition, which comprises of an effective quantity of β-amyrin acetate and a pharmaceutically acceptable vehicle for administration to humans and animals for the treatment of diabetes mellitus.

7. A pharmaceutical composition as claimed in claim 6 wherein it is combined with other known antidiabetic drugs.
